(19) United States
(12) Patent Application Publication
(10) Pub. No.: US 2014/0296971 A1
(43) Pub. Date: Oct. 2, 2014

(54) Alignment Device for Asymmetric Transcatheter Valve

(71) Applicants: Zachary Tegels, Minneapolis, MN (US); Robert Vidlund, Forest Lake, MN (US)

(72) Inventors: Zachary Tegels, Minneapolis, MN (US); Robert Vidlund, Forest Lake, MN (US)

(73) Assignee: Tendyne Holdings, Roseville, MN (US)

(21) Appl. No.: 14/255,687

(22) Filed: Apr. 17, 2014

Related U.S. Application Data
(60) Provisional application No. 61/807,697, filed on Apr. 2, 2013.

(51) Int. Cl. A61F 2/24

(52) U.S. Cl. CPC A61F 2/2436; A61F 2/2418
USPC 623/2.11

(57) ABSTRACT

This invention relates to relates to a alignment device for providing the correct valve alignment during deployment of an asymmetrical transcatheter valve while it is being deployed in a patient in need thereof, and methods of use thereof.

### CROSS REFERENCE TO RELATED APPLICATIONS

Not applicable

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

Not applicable.

### SEQUENCE LISTING INCLUDED AND INCORPORATED BY REFERENCE HEREIN

Not applicable.

### BACKGROUND

1. Field of the Invention

This invention relates to relates to a alignment device for providing the correct valve alignment during deployment of an asymmetrical transcatheter valve while it is being deployed in a patient in need thereof, and methods of use thereof.

2. Background of the Invention

Valvular heart disease and specifically aortic and mitral valve disease is a significant health issue in the US. Annually approximately 90,000 valve replacements are conducted in the US. Traditional valve replacement surgery, the orthotopic replacement of a heart valve, is an “open heart” surgical procedure. Briefly, the procedure necessitates surgical opening of the thorax, the initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated to the procedure largely due to the necessity for extra-corporeal circulation. Further, open heart surgery is often poorly tolerated in elderly patients.

Thus if the extra-corporeal component of the procedure could be eliminated, morbidities and cost of valve replacement therapies would be significantly reduced.

While replacement of the aortic valve in a transcatheter manner is the subject of intense investigation, lesser attention has been focused on the mitral valve. This is in part reflective of the greater level of complexity associated to the native mitral valve apparatus and thus a greater level of difficulty with regards to inserting and anchoring the replacement prosthesis.

Several designs for catheter-deployed (transcatheter) aortic valve replacement are under various stages of development. The Edwards SAPIEN transcatheter heart valve is currently undergoing clinical trial in patients with calcific aortic valve disease who are considered high-risk for conventional open-heart valve surgery. This valve is deployable via a retrograde transarterial (transfemoral) approach or an antegrade transapical (transventricular) approach. A key aspect of the Edwards SAPIEN and other transcatheter aortic valve replacement designs is their dependence on lateral fixation (e.g. tines) that engages the valve tissues as the primary anchoring mechanism. Such a design basically relies on circumferential friction around the valve housing or stent to prevent dislodgement during the cardiac cycle. This anchoring mechanism is facilitated by, and may somewhat depend on, a calcified aortic valve annulus. This design also requires that the valve housing or stent have a certain degree of rigidity.

At least one transcatheter mitral valve design is currently in development. The Endovalve uses a folding tripod-like design that delivers a tri-leaflet bioprosthetic valve. It is designed to be deployed from a minimally invasive transatrial approach, and could eventually be adapted to a transvenous atrial septotomy delivery. This design uses “proprietary gripping features” designed to engage the valve annulus and leaflets tissues. Thus the anchoring mechanism of this device is essentially equivalent to that used by transcatheter aortic valve replacement designs.

Various problems continue to exist in this field, including problems with insufficient articulation and sealing of the valve within the native annulus, pulmonary edema due to poor atrial drainage, perivalvular leaking around the install prosthetic valve, lack of a good fit for the prosthetic valve within the native mitral annulus, atrial tissue erosion, excess wear on the nitinol structures, interference with the aorta at the posterior side of the mitral annulus, and lack of customization, to name a few. Accordingly, there is still a need for an improved valve having a commissural sealing structure for a prosthetic mitral valve.

### BRIEF SUMMARY OF THE INVENTION

This invention relates to relates to a alignment device for providing the correct valve alignment during deployment of an asymmetrical transcatheter valve while it is being deployed in a patient in need thereof, and methods of use thereof.

In a preferred embodiment, there is provided an alignment device for deploying an asymmetric transcatheter prosthetic cardiovascular mitral valve in a patient, which comprises a prosthetic mitral valve loading tube having a lateral radio-opaque marker and a longitudinal radio-opaque marker, and an asymmetric transcatheter prosthetic mitral valve disposed within the valve loading tube, wherein the asymmetric transcatheter prosthetic mitral valve comprises an expandable stent body having valve leaflets disposed therein and an asymmetric atrial collar attached to the stent, the asymmetric atrial collar having a flattened A2 segment to reduce LVOT obstruction and the asymmetric transcatheter prosthetic mitral valve is compressed within the valve loading tube having the A2 segment of the valve aligned with the lateral radio-opaque marker and a longitudinal radio-opaque marker of the tube, wherein the lateral radio-opaque marker provides a commissure-to-commissure alignment, and wherein the longitudinal radio-opaque marker provides an A2-anterior leaflet alignment.

In another preferred embodiment, there is provided a feature wherein the valve has one or more radio-opaque markers thereon to facilitate positioning.

In another preferred embodiment, there is provided a feature where the device fits within a surgical catheter sheath having a diameter of between about 10 Fr (3.3 mm) to about 42 Fr (14 mm).

In yet another preferred embodiment, there is provided a method of providing the correct valve alignment during deployment of an asymmetrical transcatheter valve while it is being deployed in a patient in need thereof, which comprises the step of deploying an alignment device as in claim 1 from a delivery catheter being used to surgically deploy the valve into the patient in need thereof.

### BRIEF DESCRIPTION OF THE DRAWINGS

FIG. 1 is a side view of a alignment device for deploying an asymmetric transcatheter prosthetic cardiovascular mitral valve in a patient according to the present inventive subject matter.

FIG. 2 is a graphic representation of an apical transcatheter delivery of a compressed prosthetic mitral valve through a catheter to a mitral valve.

FIG. 3 is a perspective view of a loading tube being aligned with an A2 segment of a mitral valve.

FIG. 4 is perspective view of a loading tube that was successfully aligned with an A2 segment of a mitral valve and the asymmetric prosthetic mitral valve is shown as being expelled from the loading tube in the proper orientation wherein the valve's flattened A2 segment is in proper alignment with native A2 mitral valve leaflet, and the asymmetrical valve's commissural features are in proper alignment with native mitral commissures, thus reducing leaking of the deployed valve. FIG. 4 illustrates A2 and commissural alignment, but in a more anatomically correct depiction, the delivery tube would deliver the valve into the left atrium, the tube would be partially withdrawn or completely withdrawn, and the tether attached to the bottom of the valve would be used to seat the valve into the mitral annulus.

### DETAILED DESCRIPTION OF THE INVENTION

### Functions of the Alignment Device

When a transcatheter valve is delivered, the compressed valve is expelled from the delivery catheter and the valve expands to its functional structure. In the case of a prosthetic mitral valve that uses an atrial cuff in combination with a ventricular tether to seat itself within the mitral annulus, when the valve is deployed into the left atrium, the valve is expelled from the end of the delivery catheter without regard to proper alignment. This becomes especially important when using an asymmetric valve. The alignment device is used to seat the valve into the native mitral annulus in order to take advantage of the engineered anti-leakage structures developed into such asymmetric valve.

### Description of Figures

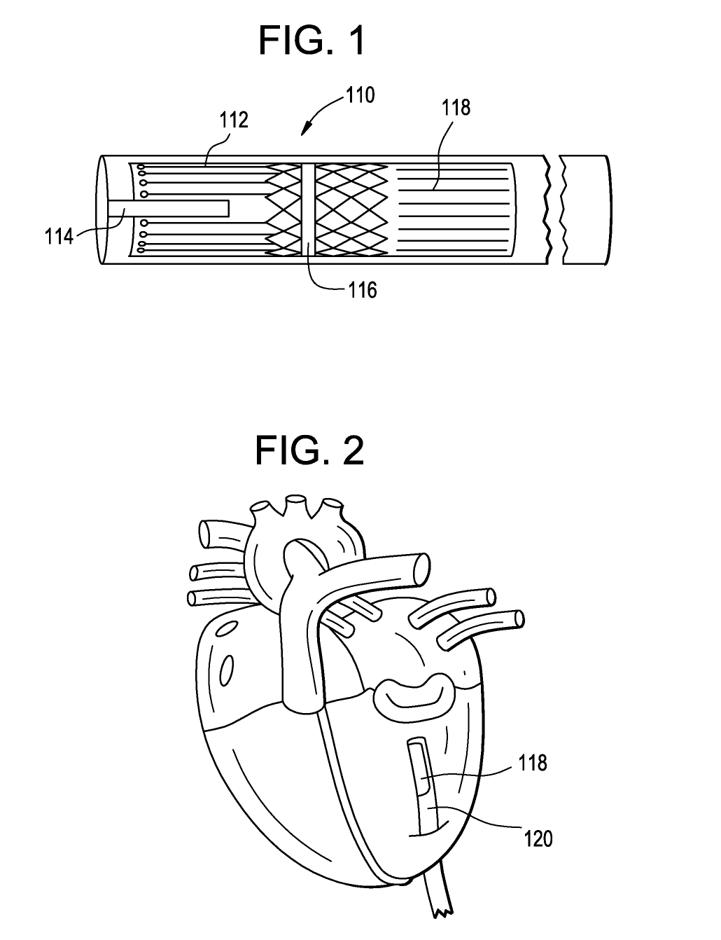

Referring now to the FIGURES, FIG. 1 is a side view of a alignment device for deploying an asymmetric transcatheter prosthetic cardiovascular mitral valve in a patient according to the present inventive subject matter. FIG. 1 shows alignment device 110 comprised of loading tube 112 and asymmetric valve 122. FIG. 1 shows longitudinal marker 114 and lateral marker 116. Longitudinal marker 114 provides A2 alignment and lateral marker 116 provides commissure-to-commissure alignment.

FIG. 2 is a graphic representation of an apical transcatheter delivery of a compressed prosthetic mitral valve 118 through a catheter 120 to a native mitral valve in need of a prosthetic.

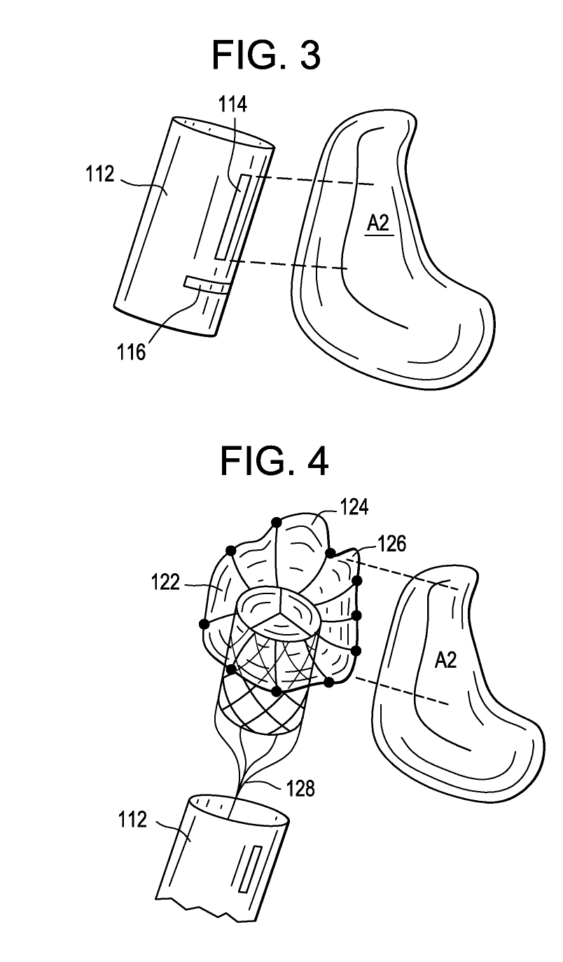

FIG. 3 is a perspective view of a loading tube 112 being aligned 114 with an A2 segment of a mitral valve. FIG. 3 also shows lateral marker 116 and illustrates how it provides commissure-to-commissure alignment.

FIG. 4 is perspective view of a loading tube 112 that was successfully aligned with an A2 segment of a mitral valve and the asymmetric prosthetic mitral valve 122 (expanded) is shown as being expelled from the loading tube 112 in the proper orientation wherein the valve's flattened A2 segment 126 is in proper alignment with native A2 mitral valve leaflet, and the asymmetrical valve's commissural features 124 are in proper alignment with native mitral commissures, thus reducing leaking of the deployed valve 122. FIG. 4 illustrates A2 and commissural alignment, but in a more anatomically correct depiction, the delivery tube 112 would deliver the valve 122 into the left atrium, the tube would be partially withdrawn or completely withdrawn, and the tether(s) 128 attached to the bottom of the valve would be used to seat the valve into the mitral annulus by pulling on it. The tether may then be secured at an appropriate location, e.g. ventricular apex.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

What is claimed is:

1. An alignment device for deploying an asymmetric transcatheter prosthetic cardiovascular mitral valve in a patient, which comprises a prosthetic mitral valve loading tube having a lateral radio-opaque marker and a longitudinal radio-opaque marker, and an asymmetric transcatheter prosthetic mitral valve disposed within the valve loading tube, wherein the asymmetric transcatheter prosthetic mitral valve comprises an expandable stent body having valve leaflets disposed therein and an asymmetric atrial collar attached to the stent, the asymmetric atrial collar having a flattened A2 segment to reduce LVOT obstruction and the asymmetric transcatheter prosthetic mitral valve is compressed within the valve loading tube having the A2 segment of the valve aligned with the lateral radio-opaque marker and a longitudinal radio-opaque marker of the tube, wherein the lateral radio-opaque marker provides a commissure-to-commissure alignment, and wherein the longitudinal radio-opaque marker provides an A2-anterior leaflet alignment.

2. The alignment device of claim 1, wherein the valve has one or more radio-opaque markers thereon to facilitate positioning.

3. The alignment device of claim 1, where the device fits within a surgical catheter sheath having a diameter of between about 10 Fr (3.3 mm) to about 42 Fr (14 mm).

4. A method of providing the correct valve alignment during deployment of an asymmetrical transcatheter valve while it is being deployed in a patient in need thereof, which comprises the step of deploying an alignment device as in claim 1 from a delivery catheter being used to surgically deploy the valve into the patient in need thereof.
